(19) United States
(12) Patent Application Publication
(10) Pub. No.: US 2022/0362167 A1
(43) Pub. Date: Nov. 17, 2022

(54) WATER-DISPERSIBLE CANNABIDIOL PRODUCT AND PREPARATION METHOD THEREFOR

(71) Applicant: GUILIN LAYN NATURAL INGREDIENTS CORP., Guilin City (CN)

(72) Inventors: Yunfei Song, Guilin City (CN); Benjun Qin, Guilin City (CN); Yongfu Xie, Guilin City (CN)

(21) Appl. No.: 17/620,008

(22) PCT Filed: Feb. 12, 2020
(86) PCT No.: PCT/CN2020/074840

§ 371 Date: Dec. 16, 2021

(30) Foreign Application Priority Data
Oct. 14, 2019 (CN) 201910972045.8

(51) Int. Cl. A61K 31/05; A61K 9/107; A61K 47/36; A61K 47/46

(52) U.S. Cl. CPC A61K 31/05; A61K 9/1075; A61K 47/36; A61K 47/46

(57) ABSTRACT

Disclosed are a water-dispersible cannabidiol product and a preparation method therefor. The water-dispersible cannabidiol product comprises a product which can be prepared by means of the following method: adding an organic phase containing cannabidiol into a water phase for emulsification; after an organic solvent in an emulsion has been removed, adding glycosylase for a glycosylation treatment; and conducting drying to prepare the water-dispersible cannabidiol product. The prepared dispersible cannabidiol product can be quickly dispersed and dissolved in an aqueous solution. The product after the glycosylation treatment is a glucoside mixture in which cannabidiol is a parent nucleus structure and in which different sugar chain structures have been introduced to phenolic hydroxyl groups.

### TECHNICAL FIELD

The present invention relates to the field of food processing, and in particular, to a water-dispersible cannabidiol product and a preparation method thereof.

### BACKGROUND ART

Antioxidant, cannabidiol (CBD), extracted from Cannabis sativa Linn. is a non-addictive active ingredient without neurotoxicity, which can antagonize the neural activity induced by THC activated cannabinoid type I receptor (CB1R). In addition, cannabidiol (CBD) has demonstrated the efficacy in various pharmacological activity, such as anti-convulsion, anti-vomiting, anti-spasm, anti-anxiety, sedation, anti-insomnia, anti-inflammatory, anti-oxidation and anti-tranquilizing effect. Cannabidiol (CBD) can be used in a variety of fields such as pharmaceuticals, healthcares, foods, and the like.

Since cannabidiol is hardly soluble in water, the absorption efficiency of human body thereof is not high, thus impeding its physiological function. Therefore, it is necessary to developing a cannabidiol product which can solve the technical problem of hardly soluble in water.

### SUMMARY

In order to solve the disadvantages of the prior art, one object of the present invention is to provide a water-dispersible cannabidiol product, which has good dispersion property when dissolved in water. Another object of the present invention is to provide a method for preparing a water-dispersible cannabidiol product, which is convenient to operate and cost-effective.

A water-dispersible cannabidiol product, comprising a product obtained by the following method: adding an organic phase containing cannabidiol to an aqueous phase for emulsification, removing organic solvents in the emulsion, then adding a glycosylase for glycosylation and drying, so as to obtain the water-dispersible cannabidiol product.

According to another object of the present invention, the present invention provides a method for preparing a water-dispersible cannabidiol product. The method comprises adding an organic phase containing cannabidiol to an aqueous phase for emulsification, removing organic solvents in the emulsion, then adding a glycosylase for glycosylation, and drying, wherein the glycosylation is carried out at a temperature of 20-60° C. for 0.5-24 hours.

Preferably, the content of the cannabidiol is 1-99% by mass.

Preferably, the organic solvent in the organic phase containing cannabidiol is one of ethyl acetate, n-hexane, n-heptane, petroleum ether, dichloromethane, benzene or chloroform, and the mass/volume ratio of the cannabidiol to the organic phase is 0.1-10:100, in kg/L.

Preferably, the aqueous phase comprises an emulsifier, and the emulsifier is one or more of sodium starch octenylsuccinate, Quillaia saponaria bark extract, maltodextrin, corn starch and algal polysaccharide.

Preferably, the volume ratio of the organic phase to the aqueous phase is 1:2-20.

Preferably, the emulsification comprises high-speed shearing emulsification and homogenous emulsification, wherein the high-speed shearing emulsification is carried out at a stirring speed of 8000-15000 r/min, for 2-30 min, and the homogenous emulsification is carried out at a pressure of 100-180 MPa.

Preferably, the cannabidiol in the emulsion has a particle diameter of ≤200 nm.

Preferably, the volume ratio of the solution obtained by removing the organic solvent in the emulsion to the glycosylase is 100: 1-10.

Preferably, the drying is spray drying, at an inlet temperature of 180-205° C., and an outlet temperature of 60-90° C.

The water-dispersible cannabidiol prepared in the present invention can be quickly dispersed and dissolved in an aqueous solution, and the solution is clear and not precipitated during a long-term storage. The product obtained after glycosylation is a glycoside mixture having mother nucleus structure of cannabidiol, and different oligosaccharide chain structures connected to the phenolic hydroxyl groups. The product of the present invention solves the technical problem that cannabidiol is insoluble in water, so that it can be widely used in the food and beverage industry. The product obtained after glycosylation can increase the stability itself, and quickly metabolize and release cannabidiol in vivo, thereby solving the technical problems such as decrease in cannabidiol content caused by decomposition, due to the instability during long-term storage of the cannabidiol product in liquid state. The powder product prepared by spray drying facilitates the transportation and storage of the product. The cannabidiol particles obtained after ultrahigh-pressure homogenization of the product have a particle size of below 200 nm, thereby improving the bioavailability of the cannabidiol product in vivo.

### DETAILED DESCRIPTION OF THE INVENTION

Hereinafter, the present invention will be further described in detail with reference to specific examples, but the embodiments of the present invention are not limited to the scope of the examples. These examples are merely intended to illustrate the invention, but not to limit the scope of the invention. Furthermore, after reading the disclosure of the present invention, those skilled in the art can make various modifications to the present invention, and these equivalent changes all fall in the scope of protection as defined in the appended claims of the present invention.

The raw materials and chemical agents used in the examples of the present invention are all conventionally commercial available, unless otherwise specified.

### Example 1

Preparation of the organic phase containing cannabidiol: 0.1 kg of the purified crystal cannabidiol with cannabidiol content of 99% by mass is taken, and 1 L of the cannabidiol solution is formulated by using n-hexane as a solvent, so as to obtain the organic phase.

Preparation of the aqueous phase: 1 kg of sodium starch octenyl succinate (Product No. HI-CAP100) is taken as an emulsifier to prepare an aqueous solution containing the emulsifier, so as to obtain the aqueous phase.

Mixing of the organic phase with the aqueous phase: under low-speed stirring (100 r/min), the organic phase is slowly poured into the 20 L of the aqueous phase, and continued to high-speed stirring (800 r/min) for 10 min, so as to obtain a homogenous mixture.

High-speed shearing emulsification: the resulting mixture is emulsified at a rotating speed of 8000 r/min for 2 min so as to obtain a crude emulsion.

Ultra-high pressure homogenization: the crude emulsion is homogenized at a pressure of 100 MPa for 15 min, so that the cannabidiol in the mixture has a particle diameter of ≤200 nm.

Solvent recovery: by using a vacuum concentration process, n-hexane is recovered at a temperature of 50° C. and a vacuum level of 0.07 MPa, and the content of the organic solvent in the product is reduced below the safe dose, so as to obtain 10 L of the cannabidiol nanodispersion with good water-dispersibility.

Glycosylation: 0.1 L of galactosyltransferase (purchased from Amano Enzyme Inc., Japan) is added to the cannabidiol dispersion system, stirred at 40° C. for 0.5 hours, then raised the temperature to 95° C., and maintained for 5 min, and removed the suspending impurities by filtration.

Spray-drying: the filtrate is pumped into a spray-drying apparatus, controlled at an inlet temperature of 180° C. and an outlet temperature of 90° C. so as to obtain the dry powder cannabidiol product with good fluidity.

### Example 2

Preparation of the organic phase containing cannabidiol: the cannabidiol oil resin with a cannabidiol content of 40% by mass is taken, and a cannabidiol solution is formulated by using ethyl acetate as a solvent, so as to obtain the an organic phase with the mass/volume ratio of the cannabidiol to the organic phase of 3:100, in kg/L.

Preparation of the aqueous phase: the sodium starch octenyl succinate and maltodextrin are selected as emulsifiers to prepare an aqueous solution containing the emulsifiers, so as to obtain the aqueous phase with mass/volume ratio of the emulsifier to the organic phase of 2:100, in kg/L.

Mixing of the organic phase and the aqueous phase: under low-speed stirring (100 r/min), the organic phase is slowly poured into the aqueous phase, controlled the volume ratio of the organic phase to the aqueous phase of 1:10, and continued to a high-speed stirring (800 r/min) for 10 min, so as to obtain a homogenous mixture.

High speed shearing emulsification: the resulting mixture is emulsified at a rotating speed of 10000 r/min for 10 min so as to obtain a crude emulsion.

Ultra-high pressure homogenization: the crude emulsion is homogenized under a pressure of 150 MPa for 15 min so that the cannabidiol in the mixture has a particle diameter of ≤200 nm.

Solvent recovery: by using a vacuum concentration process, the ethyl acetate is recovered at a temperature of 50° C. and a vacuum level of 0.08 MPa, and the content of the organic solvent in the product is reduced below the safe dose, so as to obtain the cannabidiol nanodispersion with good water dispersibility.

Glycosylation: the glycosyl transferase (purchased from Amano Enzyme Inc., Japan) is added to the cannabidiol dispersion system, with the volume ratio of the cannabidiol dispersion system to the glycosylase of 100:5, stirred at 30° C. for 8 hours, then raised the temperature to 95° C., and maintained for 5 min, and removed the suspending impurities by filtration.

Spray drying: the filtrate is pumped into a spray drying apparatus controlled at an inlet temperature of 190° C. and an outlet temperature of 70° C. so as to obtain a dry powder cannabidiol product with good fluidity.

### Example 3

Preparation of the organic phase containing cannabidiol: the cannabidiol oil resin with a cannabidiol content of 1% by mass is taken, and a cannabidiol solution is formulated by using n-heptane and petroleum ether as solvents, so as to obtain an organic phase with the mass/volume ratio of the cannabidiol to the organic phase of 8:100, in kg/L.

Preparation of the aqueous phase: three components, maltodextrin, corn starch and algal polysaccharides are selected as emulsifiers to prepare an aqueous solution containing the emulsifiers, so as to obtain the aqueous phase with the mass/volume ratio of the emulsifier to the aqueous phase of 1:100, in kg/L.

Mixing of the organic phase and the aqueous phase: under low-speed stirring (100 r/min), the organic phase is slowly poured into the aqueous phase, controlled the volume ratio of the organic phase to the aqueous phase at 1:20, and continued to high-speed stirring (800 r/min) for 10 min, so as to obtain a homogenous mixture.

High-speed shearing emulsification: the resulting mixture is emulsified at rotating speed of 15000 r/min for 30 min so as to obtain a crude emulsion. Ultra-high pressure homogenization: the crude emulsion is homogenized under a pressure of 180 MPa for 15 min, so that the cannabidiol in the mixture has a particle diameter ≤200 nm.

Solvent recovery: by using a vacuum concentration process, n-heptane and petroleum ether are recovered at a temperature of 50° C. and a vacuum level of 0.09 MPa, and the content of organic solvents in the product is reduced below the safe dose, so as to obtain the cannabidiol nanodispersion with good water dispersibility.

Glycosylation: a glycosyl transferase is added to the cannabidiol dispersion system, with the volume ratio of the cannabidiol dispersion system to the glycosylase of 100:10, stirred at 60° C. for 24 hours, then raised the temperature to 95° C., maintained for 5 min, and removed the suspending impurities by filtration.

Spray drying: the filtrate is pumped into a spray drying apparatus controlled at an inlet temperature of 205° C. and an outlet temperature of 90° C. so as to obtain a dry powder cannabidiol product with good fluidity.

### Example 4

Preparation of the organic phase containing cannabidiol: the purified crystal cannabidiol with the cannabidiol content of 85% by mass is taken, and a cannabidiol solution is formulated by using dichloromethane as a solvent, so as to obtain an organic phase with the mass/volume ratio of the cannabidiol to the organic phase of 0.1:100, in kg/L.

Preparation of the aqueous phase: five components, sodium starch octenylsuccinate (Product No. Gum2000), Quillaia saponaria bark extract (Product No. Q-Naturale), maltodextrin, corn starch and algal polysaccharides are selected as emulsifiers, to prepare an aqueous solution containing emulsifiers, so as to obtain an aqueous phase with the mass/volume ratio of the emulsifiers to the aqueous phase of 1:100, in kg/L.

Mixing of the organic phase and the aqueous phase: under low-speed stirring (100 r/min), the organic phase is slowly poured into the aqueous phase, controlled the volume ratio of the organic phase to the aqueous phase at 1:16, and continued to high-speed stirring (800 r/min) for 10 min, so as to obtain a homogenous mixture.

High-speed shearing emulsification: the resulting mixture is emulsified at rotating speed of 12000 r/min for 24 min so as to obtain a crude emulsion.

Ultra-high pressure homogenization: the crude emulsion is homogenized under a pressure of 160 MPa for 15 min, so that the cannabidiol in the mixture has a particle diameter ≤200 nm.

Solvent recovery: by using a vacuum concentration process, dichloromethane is recovered at a temperature of 50° C. and a vacuum level of 0.09 MPa, and the content of the organic solvent in the product is reduced below the safe dose so as to obtain the cannabidiol nanodispersion with good water dispersibility.

Glycosylation: a glycosyl transferase is added to the cannabidiol dispersion system, with the volume ratio of the cannabidiol dispersion system to the glycosylase of 100:7, stirred at 50° C. for 20 hours, then raised the temperature to 95° C., maintained for 5 min, and removed the suspending impurities by filtration.

Spray drying: the filtrate is pumped into a spray drying apparatus, controlled at an inlet temperature of 190° C. and an outlet temperature of 85° C. so as to obtain a dry powder cannabidiol product with good fluidity.

### Comparative Example 1

The comparative example is based on example 1, with the exception of absence of glycosylation, which is used to evaluate the difference in effect between the cannabidiol products obtained with and without the glycosylation. The preparation method is as follows:

Preparation of the organic phase containing cannabidiol: 0.1 kg of purified crystal cannabidiol with cannabidiol content of 99% by mass is taken, and 1 L of cannabidiol solution is formulated by using n-hexane as a solvent, so as to obtain an organic phase.

Preparation of the aqueous phase: 1 kg of sodium starch octenyl succinate (Product No. HI-CAP100) is taken as an emulsifier to prepare an aqueous solution containing the emulsifier, so as to obtain an aqueous phase.

Mixing of the organic phase and the aqueous phase: under low-speed stirring (100 r/min), the organic phase is slowly poured into 20 L of the aqueous phase, and continued to high-speed stirring (800 r/min) for 10 min, so as to obtain a homogenous mixture.

High-speed shearing emulsification: the resulting mixture is emulsified at rotating speed of 8000 r/min for 2 min so as to obtain a crude emulsion.

Ultra-high pressure homogenization: the crude emulsion is homogenized under a pressure of 100 MPa for 15 min, so that the cannabidiol in the mixture has a particle diameter ≤200 nm.

Solvent recovery: by using a vacuum concentration process, n-hexane is recovered at a temperature of 50° C. and a vacuum level of 0.07 MPa, and the content of the organic solvent in the product is reduced below the safe dose, so as to obtain 10 L of cannabidiol nanodispersion with good water dispersibility.

Spray drying: the cannabidiol nanodispersion is pumped into a spray drying apparatus, controlled at an inlet temperature of 180° C. and an outlet temperature of 90° C. so as to obtain a dry powder cannabidiol product.

### Comparative Example 2

This comparative example is based on example 1, with the exception of absence of the emulsification, which is used to evaluate the difference in the effect between the cannabidiol products obtained with and without emulsification. The preparation method is as follows:

Preparation of the organic phase containing cannabidiol: 0.1 kg of purified crystal cannabidiol with the cannabidiol content of 99% by mass is taken, and 1 L of the solution is formulated by using distilled water as a solvent, and 1 kg of sodium starch octenyl succinate (Product No. HI-CAP 100) is taken, continued to adding 20 L of the distilled water under low-speed stirring (100 r/min), continued to high-speed stirring (800 r/min) for 10 min, mixed uniformly, so as to obtain a suspension. After the solvent was recovered completely, 0.1 L of glycosyl transferase (purchased from Amano Enzyme Inc., Japan) is added, stirred at 40° C. for 0.5 hours, then raised the temperature to 95° C., maintained for 5 min, removed the suspending impurities by filtration.

Spray drying: the filtrate is pumped into a spray drying apparatus, controlled at an inlet temperature of 180° C., and an outlet temperature of 90° C. so as to obtain a dry powder cannabidiol product.

Water Solubility Test of Cannabidiol Product

Three parts of 500 mL of distilled water are taken. 50 mg of the cannabidiol product prepared in Example 1, Comparative Example 1 and Comparative Example 2 are added respectively at room temperature, and stirred, and recorded the time required for dissolution, with the cannabidiol as control. Experimental results show that cannabidiol is hard to dissolve in distilled water, and the solution occurs precipitation and delamination. The solutions containing cannabidiol product prepared in Example 1, Comparative Example 1 and Comparative Example 2 are clear solutions without suspending particles, wherein the product of Example 1 is completely dissolved in distilled water after 30 s, while the product of Comparative Example 1 is completely dissolved in distilled water after 90 s, and the product of Comparative Example 2 is completely dissolved in distilled water after heating, therefore the product of Example 1 has the best water solubility.

In the present invention, the wall materials such as sodium starch octenyl succinate, maltodextrin, corn starch, and algal polysaccharides are used to embed the cannabidiol, and the glycosylation is carried out, so that the influence of the external environment on the product can be reduced and the water solubility and stability of the product are significantly improved.

1. A water-dispersible cannabidiol product, characterized by comprising a product which can be prepared by the following method: adding an organic phase containing cannabidiol to an aqueous phase for emulsification, removing organic solvents in the emulsion, then adding a glycosylase for glycosylation, and drying so as to obtain the water-dispersible cannabidiol product.

2. The method for preparing the water-dispersible cannabidiol product according to claim 1, characterized in that the method comprises the steps of: adding an organic phase containing cannabidiol to an aqueous phase for emulsification, removing organic solvents in the emulsion, then adding a glycosylase for glycosylation and drying, wherein the glycosylation is carried out at a temperature of 20-60° C., for 0.5-24 hours.

3. The method for preparing the water-dispersible cannabidiol product according to claim 2, characterized in that the content of cannabidiol is 1-99% by mass.

4. The method for preparing the water-dispersible cannabidiol product according to claim 2, characterized in that the organic solvent in the organic phase containing cannabidiol is one of ethyl acetate, n-hexane, n-heptane, petroleum ether, dichloromethane, benzene or chloroform, and the mass/volume ratio of the cannabidiol to the organic phase is 0.1-10:100, in kg/L.

5. The method for preparing the water-dispersible cannabidiol product according to claim 2, characterized in that the aqueous phase comprises an emulsifier, and the emulsifier is one or more of sodium starch octenylsuccinate, Quillaia saponaria bark extract, maltodextrin, corn starch and algal polysaccharides.

6. The method for preparing the water-dispersible cannabidiol product according to claim 2, characterized in that the volume ratio of the organic phase to the aqueous phase is 1:2-20.

7. The method for preparing the water-dispersible cannabidiol product according to claim 2, characterized in that the emulsification comprises high-speed shearing emulsification and homogeneous emulsification, wherein the high-speed shearing emulsification is carried out at a stirring speed of 8000-15000 r/min, for 2-30 min, and the homogeneous emulsification is carried out at a pressure of 100-180 MPa.

8. The method for preparing the water-dispersible cannabidiol product according to claim 2, characterized in that the cannabidiol in the emulsion has a particle diameter of ≤200 nm.

9. The method for preparing the water-dispersible cannabidiol product according to claim 2, characterized in that the volume ratio of the solution obtained by removing the organic solvents in the emulsion to the glycosylase is 100: 1-10.

10. The method for preparing the water-dispersible cannabidiol product according to claim 2, characterized in that the drying is spray drying at an inlet temperature of 180-205° C., and an outlet temperature of 60-90° C.
